(19) United States
(12) Patent Application Publication
(10) Pub. No.: US 2018/0274962 A1
(43) Pub. Date: Sep. 27, 2018

(54) INJECTION DEVICE FOR LIQUID

(71) Applicants: AIT AUSTRIAN INSTITUTE OF TECNOLOGY GMBH, WIEN (AT); SEIBERSDORF LABOR GMBH, SEIBERSDORF (AT)

(72) Inventors: GERNOT SCHMID, BROMBERG (AT); MANFRED BAMMER, WIEN (AT); RENE HIRTL, WIEN (AT); ROBERT LURF, GLOGGNITZ (AT); MICHAEL MEINDL, WIMPASSING (AT); MARTIN BEISTEINER, BAD SCHOENAU (AT)

(21) Appl. No.: 15/546,486

(22) PCT Filed: Nov. 27, 2015
(86) PCT No.: PCT/AT2015/050302

§ 371 Date: Jul. 26, 2017

(30) Foreign Application Priority Data
Jan. 28, 2015 (AT) A 50058/2015

(51) Int. Cl. G01F 11/02; A61M 5/34; A61M 5/32; G01F 23/26; G01F 25/00

(52) U.S. Cl. CPC G01F 11/027; A61M 2205/14; A61M 5/3202; G01F 23/263; G01F 23/266; G01F 25/0061; G01F 25/0076; G01F 25/0084; G01F 25/0092; A61M 2205/33; A61M 2205/3317; A61M 2205/50; A61M 2205/3389; A61M 2205/70; A61M 5/347

(57) ABSTRACT

A dispensing device and a method for dispensing liquids, in particular liquid medicaments, to persons, has a liquid-filled container an opening for dispensing the liquid at one end. A holder for holding an injection needle for injection of the liquid from the container are provided in the area of the opening. A measurement system determines a measured value, and a processing unit determines a parameter value, associated with the container, based on the measured value. At least one detection element is present at the opening and connected to a needle detection circuit. The needle detection circuit measures an electrical characteristic variable that can be picked up at the output of the detection element. The measurement result is fed to a processing unit, which calculates the parameter value and keeps it available at its output.

The invention relates to a dispensing device for dispensing liquids, in particular liquid medications, according to the preamble of patent claim 1. Furthermore, the invention relates to a method for determining a physical or chemical parameter related to a container or a liquid located in the container, in particular the fill level of the liquid in the container, according to the preamble of patent claim 18.

Various dispensing devices and methods for determining fill levels in dispensing devices or other parameters which are related to the liquid in the container, which are based on capacitive detection, are known from the prior art. The essential disadvantage of all of these methods or dispensing devices is that the use of a conductive or metallic injection needle for administering the respective liquid influences the measurements based on capacitive measurement principles, so that different measurement results are provided depending on whether an injection needle is placed on the dispensing device or not.

The invention is based on the object of providing a dispensing device and a method which overcome these disadvantages.

The invention achieves this object in a dispensing device of the type mentioned at the outset using the characterizing features of patent claim 1.

The invention provides, in a dispensing device for dispensing liquids, in particular liquid medications to persons, comprising
a container filled with the liquid, which has an opening at one end for dispensing the liquid, wherein it is provided that holding means for holding an injection needle for injecting the liquid located in the container are provided in the region of the opening,
a measuring system for, in particular capacitive, determination of a measured value, and
a processing unit for determining at least one physical or chemical parameter value related to the container or the liquid located therein, in particular the fill level, on the basis of the measured value,
at least one detection element is provided in the region of the opening,
a needle detection circuit is provided, wherein the detection element is connected to the needle detection circuit,
the needle detection circuit is designed for measuring an electrical characteristic variable which can be tapped at the output of the detection element, in particular the inductance, capacitance, or conductivity,
a processing unit is provided, and the measurement result applied at the output of the needle detection circuit is supplied as a detection result to the processing unit, and
the processing unit computes the parameter as a function of the measured value and the detection result and provides it at its output.

In this case, the electrical influence of a metallic or conductive injection needle on the measurement result is advantageously substantially eliminated.

One possibility to compensate for the influence of a metallic or conductive injection needle on the measurement result provides that the processing unit compares the detection result supplied thereto to a predefined threshold value, and computes the parameter value provided at the output on the basis of the measured value, wherein it ascertains the computation rule used for computing the parameter value depending on the result of the comparison of the detection result to the predefined threshold value.

A simple procedure for numeric description and elimination of the influence of a metallic or conductive injection needle on the measurement result provides that
a first calibration function is stored in the processing unit, which specifies the relationship between the measured value and the physical or chemical parameter value related to the container or the liquid located therein upon the presence of an injection needle in the holding means,
a second calibration function is stored in the processing unit, which specifies the relationship between the measured value and the physical or chemical parameter value related to the container or the liquid located therein upon the absence of an injection needle in the holding means, and
the processing unit selects the calibration function used for the computation of the parameter as a function of the comparison of the detection result to the predefined threshold value.

Alternatively thereto, it can be provided that
the processing unit comprises a first unit and a second unit,
a first calibration function is stored in the first unit, which specifies the relationship between the measured value and the physical or chemical parameter value related to the container or the liquid located therein upon the presence of an injection needle in the holding means, wherein the first unit applies the first calibration function to the measured value applied thereto and provides the result thus obtained at its output as the first parameter value, and
a second calibration function is stored in the second unit, which specifies the relationship between the measured value and the physical or chemical parameter value related to the container or the liquid located therein upon the absence of an injection needle in the holding means, wherein the second unit applies the second calibration function to the measured value applied thereto and provides the result thus obtained at its output as the second parameter value.

It is advantageously provided in this case that,
the processing unit has a first unit for determining a first parameter value, which applies a first computation rule to the measured value, which supplies the parameter value based on the measured value upon the presence of the injection needle in the holding means,
the processing unit has a second unit for determining a second parameter value, which applies a second computation rule to the measured value, which supplies the parameter based on the measured value upon the absence of the injection needle in the holding means, and
the processing unit compares the detection result supplied thereto to a predefined threshold value and, depending on this comparison, provides either the first or second parameter ascertained by the first unit or by the second unit as the parameter at its output.

A particularly simple and advantageous compensation of the capacitive influence of the injection needle on the advantageous capacitive determination of measured values can provide that at least one pair of measuring electrodes, which are arranged in the external region, in particular applied to the wall of the container, for determining the capacitance which can be tapped between the measuring electrodes, are connected to the measuring system and this capacitance measured value is supplied as the measured value to the processing unit.

To avoid corruption of the measurement result by external fields or touching of the measuring electrodes by the measuring ends, it can be provided that in particular at least one further metal structure enclosing the measuring electrodes is provided, and preferably at least one of the metal structures is designed as an electrical shield.

To ensure rapid and secure detection of the presence or absence of an injection needle with simple mechanical construction at the same time, it can be provided that,
the attachable cap at least partially encloses the region of the dispensing device located on the side of the opening and completely encloses an injection needle possibly held by the holding means or has an opening, through which the injection needle possibly held by the holding means protrudes,
the detection element is arranged on or in the attachable cap in the region of the holding means or in the region of the injection needle, and
in particular the measuring system and the needle detection circuit are integrated into the attachable cap.

Preferred embodiments, in which the detection of the injection needle is performed by a capacitance measurement, provide that
the detection element is designed as a conductive detection electrode arranged in the region of the opening,
the detection electrode is electrically coupled to a needle detection circuit.

In particular, it can be provided in this case that at least one of the measuring electrodes of the measuring system functions as the metallic structure.

One particularly preferred possibility for detecting the presence of an injection needle by capacitance measurement provides that
the detection electrode and optionally the metallic structure, in particular in the form of a further detection electrode, are arranged in or on the attachable cap in the region of the holding means or in the region of the injection needle, and/or
the detection electrode and optionally the metallic structure, in particular in the form of a further detection electrode, are arranged in the region of the holding means for the injection needle on the outer wall of the container, and/or
the detection electrode and optionally the metallic structure, in particular in the form of a further detection electrode, are arranged in the region of the holding means for the injection needle in the external region of the dispensing device, spaced apart from the container.

A further simple detection of an injection needle provides that,
the detection element is formed by a switch,
the needle detection circuit is designed to measure the electrical conductivity between the contacts of the switch,
the switch is mechanically actuated by the presence of an injection needle, and
the switch is electrically coupled to the processing unit.

In this case, it is advantageous for the detection if an electrical signal is supplied as the detection result to the threshold value comparator of the processing unit via the switch, on the basis of the actuation of the switch.

Alternatively, the presence of an injection needle may also be determined inductively, in particular in that
the detection element is formed by a conductor arrangement or coil, the inductance of which has a different value upon the presence of an injection needle than upon the absence of the injection needle,
the conductor arrangement or the coil is electrically coupled to the needle detection circuit designed as an inductance measuring circuit,
the needle detection circuit is provided for measuring the inductance of the conductor arrangement or coil, wherein the detection result applied at the output of the needle detection circuit is supplied in the form of an inductance measurement result to the processing unit,
the processing unit computes the parameter as a function of the measurement result and the detection result and provides it at its output, wherein in particular
the conductor arrangement or coil are arranged in the region of the holding means for the injection needle on the outer wall of the container, or
the conductor arrangement or coil are arranged in the region of the holding means for the injection needle in the external region of the dispensing device, spaced apart from the container.

Advantageous arrangements of the coils provide that the coil is arranged such that upon the presence of the injection needle, the turns of the coil at least partially wrap around it.

In this case, it can also be provided that the conductor arrangement or coil are arranged in or on the attachable cap in the region of the holding means or in the region of the injection needle, wherein in particular the coil is arranged such that upon the presence of the injection needle and cap attached to the dispensing device, the turns of the coil at least partially wrap around the injection needle.

Furthermore, the presence of an injection needle can be determined in that the detection element is formed by two contact elements, which, upon the presence of an electrically conductive injection needle, are in electrical contact therewith, wherein in particular an electrical signal is supplied to the threshold value comparator of the processing unit as the detection result via the electrical connection between the contact elements and the electrically conductive needle.

It is of particular hygienic advantage in the detection of the injection needle if
an electrically conductive coating is provided on a holding part captively connected to the injection needle, and
two detection elements in the form of contact elements are provided, which are in electrical contact upon the presence of an injection needle having the electrically conductive coating, wherein in particular
an electrical signal is supplied to the threshold value comparator of the processing unit as the detection result via the electrical connection between the contact elements and the electrically conductive coating, and/or
the contact elements are arranged in or on the attachable cap in the region of the holding means or in the region of the injection needle.

Furthermore, according to the invention, in a method for determining a physical or chemical parameter related to a container or a liquid located in the container, in particular the fill level of the liquid in the container,
wherein the container has an opening at one end for dispensing the liquid, and wherein holding means for holding an injection needle for injecting the liquid located in the container are provided in the region of the opening and
wherein a measured value is ascertained in the region of the container, it is provided that
by means of a detection element located in the region of the opening, a detection result dependent on the presence of an injection needle is ascertained, in particular by ascertaining the capacitance between a metallic detection electrode located in the region of the opening and a further metallic structure arranged in or on the dispensing device,
the detection result is compared to a threshold value and the presence or the absence of an injection needle in the holding means is established depending on the threshold value comparison, wherein
upon detection of the presence of an injection needle, a first parameter is applied to the measured value by applying a first computation rule, which supplies, on the basis of the measured value, the parameter value upon the presence of the injection needle in the holding means, and the first parameter thus ascertained is provided as the parameter value, and
upon detection of the absence of an injection needle, a second parameter value is applied to the measured value by applying a second computation rule, which supplies, on the basis of the measured value, the parameter value upon the absence of the injection needle in the holding means, and the second parameter thus ascertained is provided as the parameter value.

In this way, a method is provided in a simple manner, using which a physical parameter, which is related to the liquid located in the container, can be ascertained, without this having been disadvantageously influenced by the presence or absence of the injection needle.

A simple procedure for numeric description and elimination of the influence of a metallic or conductive or magnetically active injection needle on the measurement result provides that
a first calibration function is predefined, which specifies the relationship between the measured value and the physical or chemical parameter value related to the container or the liquid located therein upon the presence of an injection needle in the holding means,
a second calibration function is predefined, which specifies the relationship between the measured value and the physical or chemical parameter value related to the container or the liquid located therein upon the absence of an injection needle in the holding means, and
upon detection of the presence of the one injection needle, the first calibration function is applied to the measured value and the result thus obtained is provided as the parameter, and
upon detection of the absence of the one injection needle, the second calibration function is applied to the measured value and the result thus obtained is provided as the parameter value.

To obtain a value which is particularly simple to detect electrically, it can be provided that the capacitance which can be tapped between two measuring electrodes, which are arranged in the external region, in particular applied to the wall of the container, is used as the measured value.

Multiple preferred exemplary embodiments of the invention are described in greater detail on the basis of the following figures of the drawings:

FIG. 1 shows a first embodiment of a dispensing device according to the invention from the side.

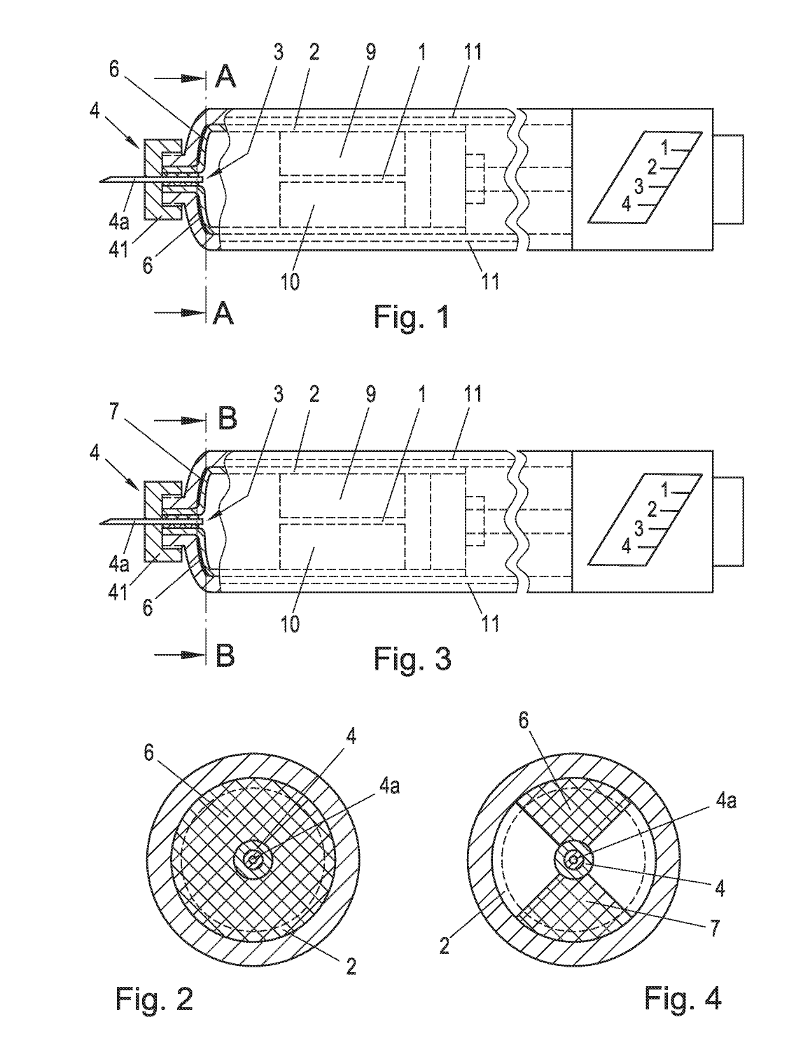

FIG. 2 shows a section through the embodiment of the invention illustrated in FIG. 1 along section edge A-A.

FIG. 3 shows a dispensing device according to a second embodiment of the invention.

FIG. 4 shows a section through the embodiment of the invention illustrated in FIG. 3 along section edge B-B.

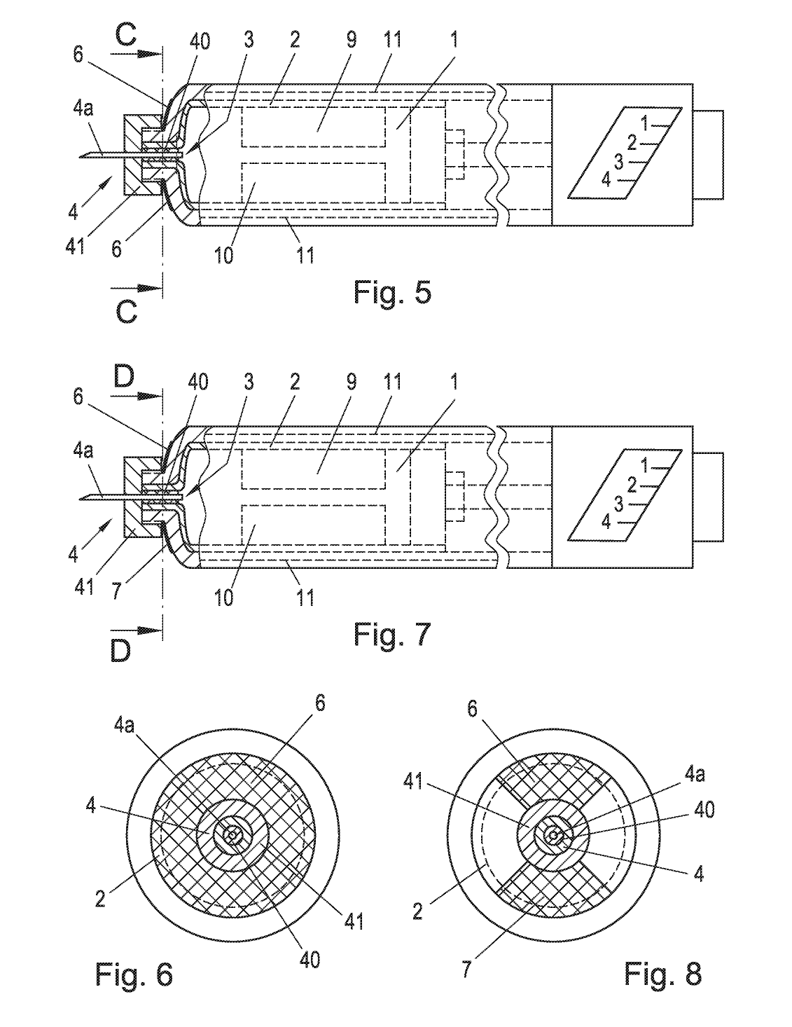

FIG. 5 shows a third embodiment of a dispensing device according to the invention from the side.

FIG. 6 shows a section through the embodiment of the invention illustrated in FIG. 5 along section edge C-C.

FIG. 7 shows a fourth embodiment of a dispensing device according to the invention from the side.

FIG. 8 shows a section through the embodiment of the invention illustrated in FIG. 7 along section edge D-D.

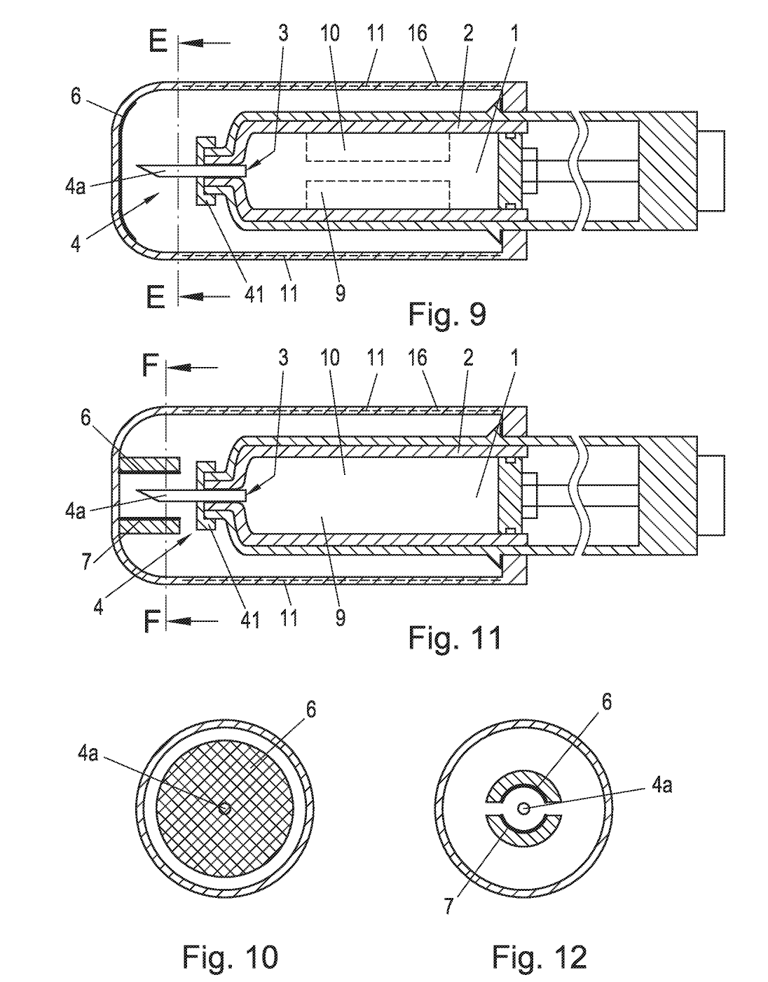

FIG. 9 shows a fifth embodiment of a dispensing device according to the invention from the side.

FIG. 10 shows a section through the embodiment of the invention illustrated in FIG. 9 along section edge E-E.

FIG. 11 shows a sixth embodiment of a dispensing device according to the invention from the side.

FIG. 12 shows a section through the embodiment of the invention illustrated in FIG. 11 along section edge F-F.

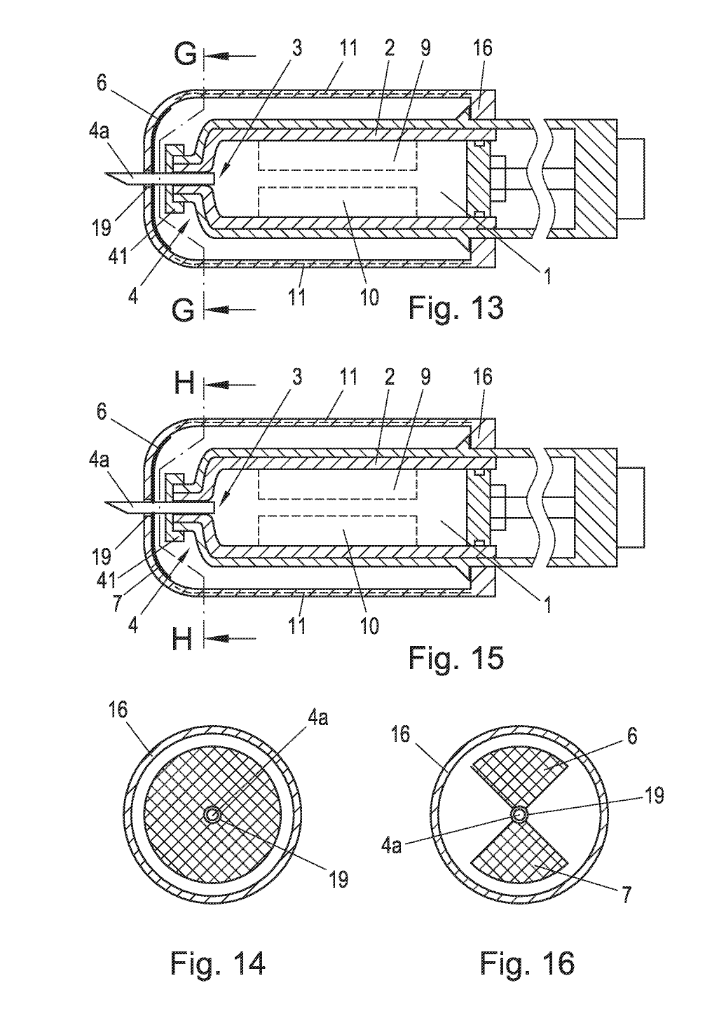

FIG. 13 shows a seventh embodiment of a dispensing device according to the invention from the side.

FIG. 14 shows a section through the embodiment of the invention illustrated in FIG. 13 along section line G-G.

FIG. 15 shows an eighth embodiment of a dispensing device according to the invention from the side.

FIG. 16 shows a section through the embodiment of the invention illustrated in FIG. 15 along section edge H-H.

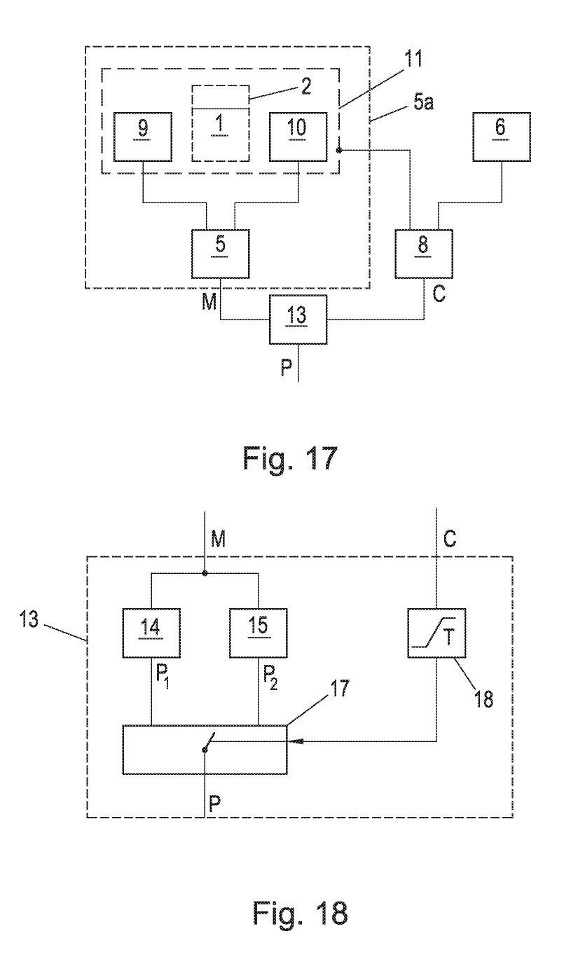

FIG. 17 schematically shows the interconnection of the measuring system of the processing unit and the measuring circuit.

FIG. 18 shows the processing unit illustrated in FIG. 17 in detail.

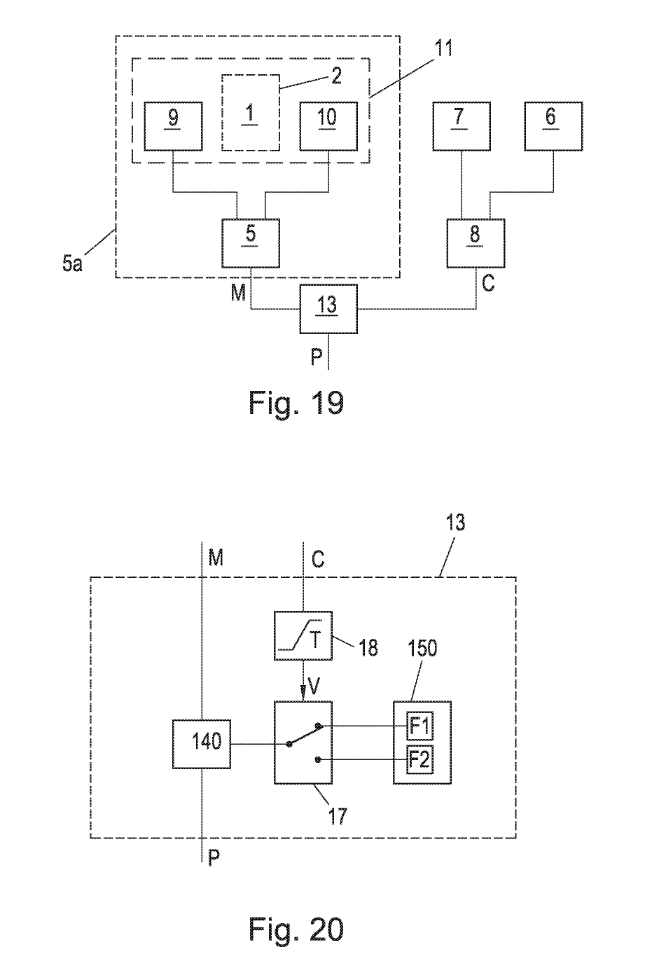

FIG. 19 schematically shows an alternative interconnection of the measuring system of the processing unit and the measuring circuit.

FIG. 20 shows an alternative embodiment of the processing unit.

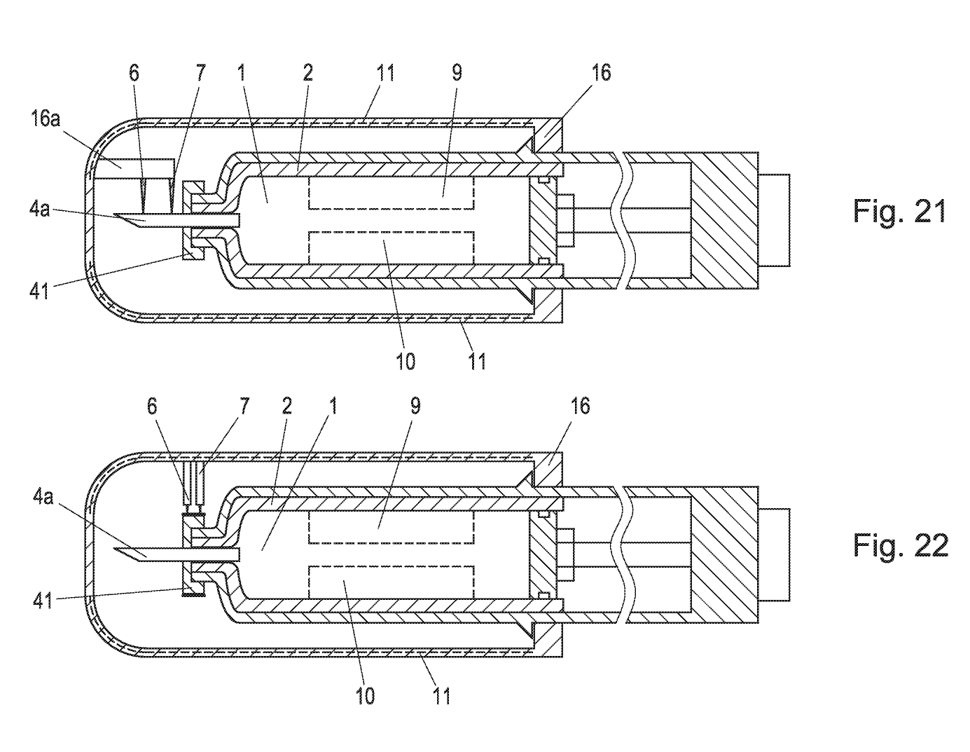

FIGS. 21 to 23 show alternative embodiments, in which the presence of an injection needle is detectable by changing a conductivity.

FIGS. 24 to 26 show further alternative embodiments having inductive detection.

FIG. 1 shows a first embodiment of a dispensing device according to the invention for dispensing liquids 1. This liquid 1 is located in a container 2, which has an opening 3 for dispensing the liquid at one end. It is provided in this case that holding means 4 for holding an injection needle 4a are provided in the region of the opening 3. The injection needle 4a is used for injecting the liquid 1 located in the container 2.

In conjunction with the invention, all required units of the dispensing device, which hold the injection needle 4a in its position in relation to the opening 3 of the container 2, can be used as a holding means 4 for holding the injection needle 4a. These can be in particular a septum 40, which is arranged in the opening 3 of the container 2, and moreover closes it, and can be pierced by means of an injection needle 4a. A combination of an external thread in the region of the opening with a holding part 41, which is fixedly connected to the injection needle 4a, having an internal thread can also function as the holding means 4, by which the injection needle 4a is held in its position.

FIG. 1 furthermore shows two measuring electrodes 9, 10 of a measuring arrangement 5a, by means of which a measured value M (FIG. 17) can be determined in the region of the container 2. On the basis of this measured value M, it is possible in principle to ascertain a physical or chemical parameter P related to the container 2 or the liquid 1 located therein. Such a parameter P is preferably the fill level of the liquid 1 in the container 2. Alternatively, there is also the possibility of ascertaining the type of the liquid itself, its temperature, its pressure, or similar values on the basis of a capacitive or other sensor measurement in the region of the container 2.

The interconnection shown in FIG. 17 and the individual components are all arranged on the dispensing device. A measuring system 5 is connected to the two measuring electrodes 9, 10. A measured value M is applied to the output of the system, on the basis of which the physical or chemical parameter P can be ascertained.

To avoid a corruption of measurement results, which is caused externally as a result of physical touching of the dispensing device, in the present exemplary embodiment, a shield 11 is preferably provided, which is arranged outside the two measuring electrodes 9, 10 and encloses them and the container 2.

In the present embodiment, a detector element 6 in the form of a detection electrode 6 is arranged on the container 2. As is apparent from FIG. 1, the detection electrode 6 is a metallic plate or loop, which is arranged between the container 2 and the housing wall, and which is used as a capacitive electrode. As shown in FIG. 17, the detection electrode 6 is connected to a needle detection circuit 8 in the form of a capacitance measuring circuit 8. The second terminal of the capacitance measuring circuit 8 can be connected to a metallic structure 7, in particular to one of the measuring electrodes 9, 10, the shield 11, the ground of the measuring system 5, or a further detection electrode 7 (FIG. 3 or FIG. 19). In the embodiment shown in FIG. 17, the second terminal of the capacitance measuring circuit 8 is electrically conductively connected to the shield 11. If an injection needle 4a is present in the region of the holding means 4 in the region of the opening 3, a detection result in the form of a capacitance measurement result C is ascertained between the detection electrode 6 and the shield 11. If the injection needle 4a is removed from the holding means 4, a capacitance measurement result C deviating therefrom is thus ascertained using the capacitance measuring circuit 8. Depending on the capacitance measurement result C, which is provided at the output of the capacitance measuring circuit 8, it is possible to recognize whether the injection needle 4a is located in the holding means 4 in the region of the opening 3 or not. This can advantageously be ascertained by means of a threshold value comparison, wherein the threshold value T is set in particular between a previously determined capacitance upon the presence of the injection needle 4a and a previously determined capacitance upon the absence of the injection needle 4a, which deviates therefrom.

Both the measured value M and also the capacitance measurement result C are supplied to a processing unit 13.

The processing unit 13, shown in detail in a specific embodiment in FIG. 18, has a threshold value comparator 18. The capacitance measurement result C is supplied to the threshold value comparator 18, which, on the basis of the previously predefined threshold value T, makes a statement about whether the capacitance measurement result exceeds or falls below this threshold value T. On the basis of the comparison of the threshold value T to the capacitance measurement result C, it can be ascertained whether an injection needle 4a is located in the holding means 4 or not. The comparison value V is provided at the output of the threshold value comparator 18.

In the embodiment shown in FIG. 18, the processing unit 13 furthermore has two units 14, 15, to each of which the measured value M is supplied. The processing unit 13 has a first unit 14 for determining a first parameter value P1 and a second unit 15 for determining a second parameter value P2.

Depending on whether the injection needle 4a is plugged into the holding means 4 or not, the computation of the parameter P on the basis of the measured value M is performed according to different computation rules. There is a first computation rule, which supplies the parameter P on the basis of the measured value M upon the presence of the injection needle 4a in the holding means 4. There is a second computation rule, which supplies the parameter P on the basis of the measured value M upon the absence of the injection needle 4a in the holding means 4. On the basis of the measured value M, two parameter values P1, P2 are determined, of which the first parameter value P1 is computed according to the first computation rule and the second parameter value P2 is computed according to the second computation rule.

The two computation rules are executed in the two units 14, 15. The first unit 14 executes the first computation rule. The first parameter value P1 is applied at its output, which correctly reflects the parameter P when the injection needle 4a is located in the holding means 4. The second unit 15 executes the second computation rule. The second parameter value P2 is applied at its output, which correctly reflects the parameter P when the injection needle 4a is not located in the holding means 4 or the holding means 4 are free of an injection needle 4a.

The two parameter values P1 and P2 are supplied to a selection unit 17. The comparison value V of the threshold value comparator 18, which is dependent on the capacitance measured value C, is used to control the selection unit 17.

If an injection needle 4a is present in the holding means 4, the first parameter value P1 is provided as the parameter P at the output of the comparison unit 13 or the selection unit 17, otherwise the second parameter value P2 is provided at the output of the processing unit 13 as the parameter P.

Particularly advantageously, calibration functions F1, F2 can respectively be stored both in the first unit 14 and also in the second unit 15, which describe the respective first and/or second computation rule as a relationship between the measured value M and the physical and chemical parameter value P related to the container or the liquid located therein upon the presence or absence of an injection needle 4a in the holding means 4.

The first unit 14 applies the first calibration function F1 to the measured value M applied thereto. It provides the result thus obtained at its output as the first parameter value P1. The second unit 15 applies the second calibration function F2 to the measured value M applied thereto. It provides the result thus obtained at its output as the second parameter value P2.

Alternatively to the embodiment of the processing unit 13 shown in FIG. 18, the embodiment of the processing unit 13 shown in FIG. 20 is also possible. In this embodiment, the processing unit 13 comprises only one computation unit 140, and a memory 150, which provides two different calibration functions F1 and F2. According to FIG. 20, the respective matching calibration function is supplied to the computation unit 140, as a function of the threshold value comparison result V. Upon supply of the calibration function F1, a parameter value is computed by the computation unit 140 and provided at the output of the processing unit 13, which correctly reflects the parameter P when an injection needle 4a is present in the holding means 4. Upon supply of the calibration function F2, a parameter value is computed by the computation unit 140 and provided at the output of the processing unit 13, which correctly reflects the parameter P when no injection needle 4a is present in the holding means 4.

FIGS. 3 and 4 show a second embodiment of the invention in greater detail. Therein, two detection electrodes 6, 7 which are diametrically opposite to one another at the opening are provided in the region of the opening 3; on the basis of the capacitance C between these two detection electrodes 6, 7, the presence or absence of an injection needle 4a in the holding means 4 in the region of the opening 3 can be established. Otherwise, this embodiment corresponds to the first embodiment of the invention. The specific arrangement of the individual components used for the measurement corresponds to the arrangement of the first embodiment of the invention shown in FIG. 17 with the difference, shown in FIG. 19, that both detection electrodes 6, 7 are connected to the measuring system 5.

The third embodiment of the invention shown in FIGS. 5 and 6 essentially corresponds to the first embodiment of the invention shown in FIGS. 1 and 2. The essential difference is that the detection electrode 6 is arranged spaced apart from the container 2 in the external region of the dispensing device. The detection electrode 6 is designed in this case in the form of an electrode ring arranged in the external region of the container 2. As also in the first exemplary embodiment of the invention, it is possible to connect one of the measuring electrodes 9, 10, the shield 11, the ground of the measuring system 5, or another metallic structure to the second input of the capacitance measuring circuit 8.

The fourth embodiment of the invention shown in FIGS. 7 and 8 essentially corresponds to the second embodiment of the invention shown in FIGS. 3 and 4. The essential difference between the second and fourth embodiments of the invention is that the two detection electrodes 6, 7 are arranged spaced apart from the container 2 in the external region of the dispensing device.

A fifth embodiment of the invention is shown in greater detail in FIGS. 9 and 10, which corresponds to the first embodiment of the invention except for the differences described hereafter. The dispensing device has a cap 16. A shield 11 can optionally be provided in this cap 16. The detection electrode 6 is located in the end region of the cap 16, which completely encloses the needle 4a in the present case. As also in the first and third exemplary embodiments of the invention, a capacitance can be performed between the detection electrode 6 and a further metallic structure 7, 9, 10, 11, in particular one of the measuring electrodes 9, 10 of the shield 11 or the ground of the measuring system.

The sixth embodiment of the invention shown in FIGS. 11 and 12 corresponds to the second and fourth embodiments of the invention shown in FIGS. 3 and 4, wherein, in contrast thereto, the two detection electrodes are arranged in the interior of the cap 16. A shield 11 can optionally be provided in this cap 16. The detection electrodes 6, 7 are located in the end region of the cap 16 and are diametrically opposite to one another in the region of the injection needle 4a.

In a seventh embodiment of the invention, shown in FIGS. 13 and 14, which essentially corresponds to the fifth embodiment of the invention, the cap 16 has an opening 19, through which the injection needle 4a passes. The detection electrode 6 is designed as ring-shaped in the inner end region of the cap 16 and encloses the opening 19 penetrated by the injection needle 4a.

FIGS. 15 and 16 show an eighth embodiment of the invention, which essentially corresponds to the seventh embodiment of the invention. An opening 19, through which the injection needle 4a can pass, is provided in the cap 16 in this case. The two detection electrodes 6, 7 are located in the region of the opening 19 provided for the injection needle 4a.

FIG. 21 shows an alternative ninth embodiment of the invention, which essentially corresponds to the sixth embodiment of the invention. The detection of the presence or absence of the injection needle 4a is not performed in this case via two capacitive electrodes, however, but rather via two electrodes 6, 7, which protrude from a shared base body 16a, which is arranged on the cap 16, in the direction of the injection needle 4a. If an electrically conductive injection needle 4a is present, it thus closes the contact between the two electrodes 6, 7. The needle detection circuit 8, which measures the conductivity between the two electrodes 6, 7 in this case, detects a conductivity between the electrodes 6, 7 upon the presence of an injection needle 4a, but no conductivity is detected upon the absence of the injection needle 4a. The conductivity is provided as the detection result C at the output of the needle detection circuit 8.

FIG. 22 shows a tenth embodiment of the invention, which essentially corresponds to the ninth embodiment of the invention. A combination of an external thread in the region of the opening with a holding part 41, which is fixedly connected to the injection needle 4a, having an internal thread is also used as the holding means 4, by which the injection needle 4a is held in its position. A conductive layer is arranged in the external region of the holding part bearing the internal thread.

Two measuring electrodes 6, 7 protrude from the internal region of the cap 16 or from a base body connected to the cap. If an electrically conductive injection needle 4a is present, a conductive contact thus also results between the measuring electrodes 6, 7 with the conductive layer arranged on the holding part. The conductive layer thus closes the contact between the two electrodes 6, 7.

The needle detection circuit 8, which measures the conductivity between the two electrodes 6, 7 in this case, detects a conductivity between the electrodes 6, 7 upon the presence of an injection needle 4a, but no conductivity is detected upon the absence of the injection needle 4a. The conductivity is provided as the detection result C at the output of the needle detection circuit 8.

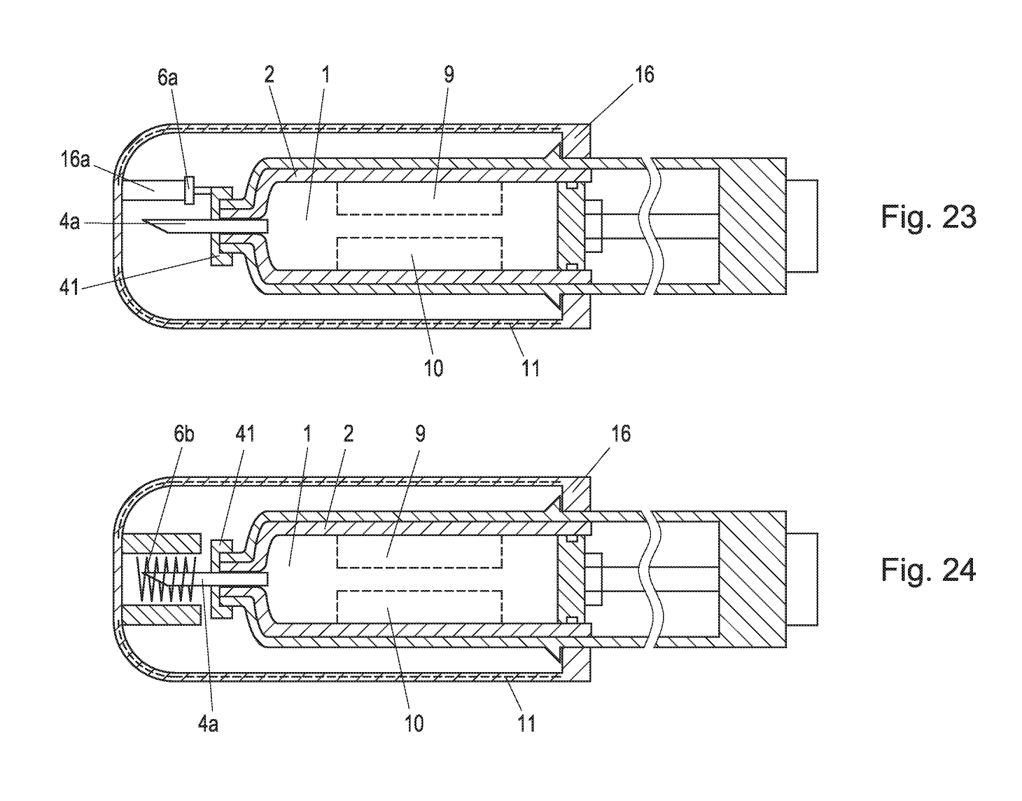

FIG. 23 shows an eleventh embodiment of the invention, which essentially corresponds to the tenth embodiment of the invention.

A combination of an external thread in the region of the opening with a holding part 41, which is fixedly connected to the injection needle 4a, having an internal thread is also used as the holding means 4, by which the injection needle 4a is held in its position. A feeler 6a protrudes from the internal region of the cap 16. The holding part bearing the internal thread protrudes into the stroke range of the feeler 6a and actuates it.

The needle detection circuit 8 is connected downstream from the feeler 6a and therefore detects whether an injection needle 4a is present or not. The conductivity is provided as the detection result C at the output of the needle detection circuit 8.

FIG. 24 shows a twelfth embodiment of the invention, which essentially corresponds to the sixth embodiment of the invention. Instead of the detection electrodes 6 and 7, a detection coil 6b is provided, which is arranged in a region into which—if present—the injection needle 4a protrudes, and the two terminals of which and the needle detection circuit are connected. Because of the conductive and/or magnetic properties of the injection needle 4a, the detection coil 6b has a different inductance upon the presence of an injection needle 4a than upon the absence of an injection needle 4a.

The needle detection circuit 8, which measures the inductance of the detection coil 6b in this case, provides this inductance as the detection result C at its output.

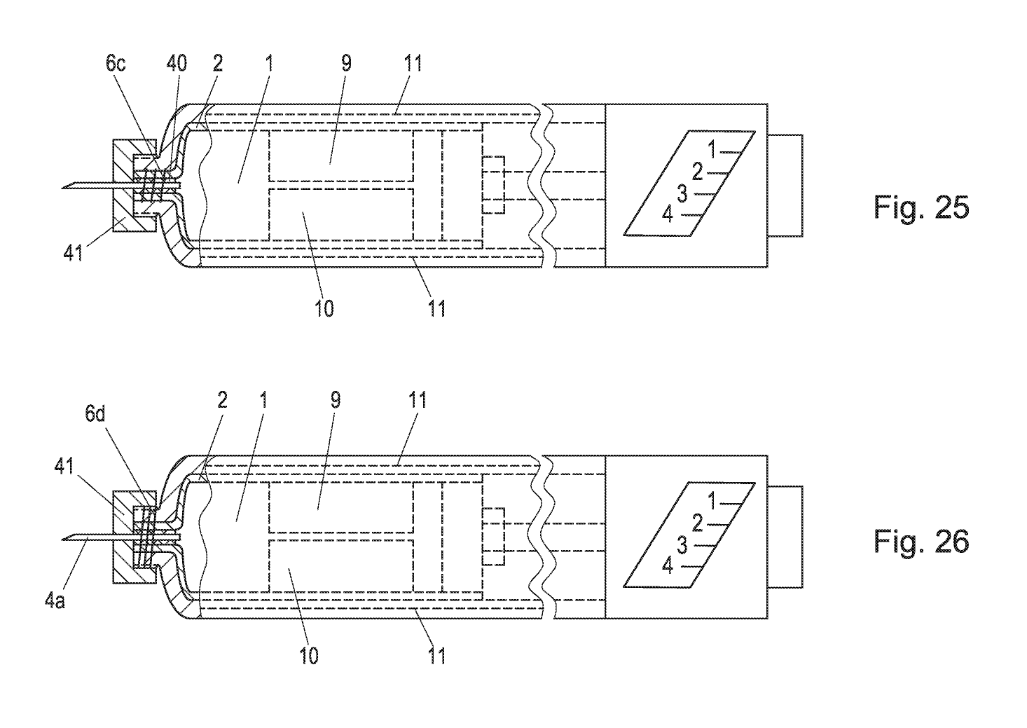

FIG. 25 shows a thirteenth embodiment of the invention, which essentially corresponds to the twelfth embodiment of the invention. In contrast to FIG. 23, in FIG. 24 the detection coil 6c is arranged between the container and the housing inner wall of the dispensing device. If an injection needle 4a is inserted into the opening 3 or guided through the septum, the inductance measured at the detection coil 6c as the detection result C changes because of the different electrical and/or magnetic properties of the injection needle 4a.

FIG. 26 shows a fourteenth embodiment of the invention, which essentially corresponds to the thirteenth embodiment of the invention. In contrast to FIG. 24, in FIG. 25 the detection coil 6d, spaced apart from the container 2, is arranged between the dispensing device and the part of the holding means 4 bearing the internal thread. If an injection needle 4a is inserted into the opening 3 or guided through the septum, the inductance measured at the detection coil 6d as the detection result C changes because of the different electrical and/or magnetic properties of the injection needle 4a.

1-20. (canceled)

21. A dispensing device for dispensing a liquid, the dispensing device comprising:
a container to be filled with the liquid and having an opening at one end for dispensing the liquid, a holding device for holding an injection needle for injecting the liquid from the container disposed at said opening;
a measuring system for determining a measured value;
a processing unit for determining at least one physical or chemical parameter value related to the container or the liquid located therein based on the measured value,
a needle detection circuit;
at least one detection element disposed at said opening and connected to said needle detection circuit;
said needle detection circuit being configured for measuring an electrical characteristic variable to be tapped at an output of said detection element (inductance, capacitance, or conductivity);
a processing unit configured to receive a measurement result at the output of said needle detection circuit as a detection result, to compute the parameter value in dependence on the measured value and the detection result, and to output the parameter value at an output thereof.

22. The dispensing device according to claim 21, wherein said processing unit is configured to compare the detection result supplied thereto with a predefined threshold value, and to compute the parameter value provided at the output based on the measured value, wherein said processing unit is configured to ascertain a computation rule used for computing the parameter value depending on a result of the comparison of the detection result with the predefined threshold value.

23. The dispensing device according to claim 21, wherein:
said processing unit has a first calibration function stored therein which specifies a relationship between the measured value and the physical or chemical parameter value related to the container or the liquid in the container upon a presence of an injection needle in the holding device;
said processing unit has a second calibration function stored therein which specifies the relationship between the measured value and the physical or chemical parameter value related to the container or the liquid in the container upon an absence of an injection needle in the holding device; and
said processing unit is configured to select the calibration function used for the computation of the parameter in dependence on the comparison of the detection result to the predefined threshold value.

24. The dispensing device according to claim 21, wherein:
said processing unit comprises a first unit and a second unit;
said first unit has a first calibration function stored therein which specifies a relationship between the measured value and the physical or chemical parameter value related to the container or the liquid in the container upon a presence of an injection needle in the holding device;
said first unit is configured to apply the first calibration function to the measured value applied thereto and to provide a result thus obtained at its output as a first parameter value;
said second unit has a second calibration function stored therein which specifies a relationship between the measured value and the physical or chemical parameter value related to the container or the liquid in the container upon an absence of an injection needle in the holding device; and
said second unit is configure to apply the second calibration function to the measured value applied thereto and to provide a result thus obtained at its output as the second parameter value.

25. The dispensing device according to claim 21, wherein:
said processing unit has a first unit for determining a first parameter value, which applies a first computation rule to the measured value, which supplies the parameter value on the basis of the measured value upon the presence of the injection needle in the holding device;
said processing unit has a second unit for determining a second parameter value, which applies a second computation rule to the measured value, which supplies the parameter on the basis of the measured value upon the absence of the injection needle in the holding device; and
said processing unit is configured to compare a detection result supplied thereto to a predefined threshold value and, depending on a result of the comparison, provides either the first parameter ascertained by the first unit or the second parameter ascertained by the second unit as the parameter at its output.

26. The dispensing device according to claim 21, which comprises at least one pair of measuring electrodes disposed on an exterior of said container, for determining a capacitance between said measuring electrodes, and connected to said measuring system, wherein a capacitance measured value is supplied as the measured value to said processing unit, and which further comprises at least one further metal structure enclosing said measuring electrodes and forming an electric shield for said measuring electrodes.

27. The dispensing device according to claim 21, comprising an attachable cap at least partially enclosing a region of the dispensing device located on a side of said opening and completely enclosing an injection needle possibly held by said holding device or having an opening, through which the injection needle possibly held by the holding device protrudes, and wherein:
said detection element is arranged on or in said attachable cap in a region of the holding device or in a region of the injection needle; and
said measuring system and said needle detection circuit are integrated into said attachable cap.

28. The dispensing device according to claim 21, wherein:
said detection element is a conductive detection electrode arranged at said opening;
said detection electrode is electrically coupled to a needle detection circuit;
said needle detection circuit is configured for measuring a capacitance between said detection electrode and a further metallic structure arranged in or on the dispensing device, wherein a capacitance measurement result applied at the output of said needle detection circuit is supplied as the detection result to said processing unit; and
said processing unit is configured to compute the parameter as a function of the measurement result and the detection result and to provide the parameter at an output thereof.

29. The dispensing device according to claim 28, wherein at least one of the following is true:
said further metallic structure is a further detection electrode at said opening; or
a ground of said measuring system functions as said further metallic structure; or
at least one of said measuring electrodes of said measuring system functions as said further metallic structure; or
a further metal structure or shield functions as said further metallic structure; or
a metal ring provided on an exterior of said container at said opening functions as said further metallic structure.

30. The dispensing device according to claim 28, wherein one or more of the following is true:
said detection electrode and optionally said further metallic structure, are arranged in or on said attachable cap in a region of said holding device or in a region of said injection needle; and/or
said detection electrode and optionally said further metallic structure are arranged in the region of said holding device for said injection needle on the outer wall of said container; and/or
said detection electrode and optionally said further metallic structure are arranged in the region of said holding device for said injection needle on the exterior of the dispensing device, spaced apart from said container.

31. The dispensing device according to claim 21, wherein:
said detection element is a switch with contacts, said needle detection circuit is configured for measuring an electrical conductivity between the contacts of said switch;
said switch is mechanically actuated by a presence of the injection needle; and
said switch is electrically coupled to said processing unit.

32. The dispensing device according to claim 31, wherein an electrical signal is supplied as the detection result to a threshold value comparator of said processing unit via said switch on the basis of an actuation of said switch.

33. The dispensing device according to claim 21, wherein:
said detection element is a conductor arrangement or coil, the inductance of which has a different value upon a presence of an injection needle than upon an absence of the injection needle; and
said conductor arrangement or coil is electrically coupled to said needle detection circuit which is an inductance measuring circuit; and
said needle detection circuit is configured for measuring an inductance of said conductor arrangement or coil, wherein the detection result applied at the output of the needle detection circuit is supplied in the form of an inductance measurement result to the processing unit; and
said processing unit computes the parameter as a function of the measurement result and the detection result and provides the parameter at its output; and
said conductor arrangement or coil are arranged in a region of said holding device for the injection needle on an outer wall of said container; or
said conductor arrangement or coil are arranged in the region of said holding device for the injection needle exterior of the dispensing device, spaced apart from said container.

34. The dispensing device according to claim 33, wherein said coil is arranged such that, when said injection needle is present, turns of said coil at least partially wrap around the injection needle.

35. The dispensing device according to claim 33, wherein said conductor arrangement or coil are arranged in or on said attachable cap in a region of said holding device or in the region of said injection needle, wherein in particular said coil is arranged such that, upon a presence of said injection needle and said cap attached to the dispensing device, turns of said coil at least partially wrap around said injection needle.

36. The dispensing device according to claim 21, wherein said detection element comprises two contact elements, which, when an electrically conductive injection needle is present, are in electrical contact therewith, wherein an electrical signal is supplied to a threshold value comparator of said processing unit as the detection result via the electrical connection between said contact elements and said electrically conductive needle.

37. The dispensing device according to claim 21, wherein:
an electrically conductive coating is provided on a holding part captively connected to said injection needle; and
two detection elements in the form of contact elements are provided, which are in electrical contact upon the presence of an injection needle having the electrically conductive coating; and
an electrical signal is supplied to the threshold value comparator of the processing unit as the detection result via the electrical connection between the contact elements and the electrically conductive coating; and/or
said contact elements are arranged in or on an attachable cap at said holding device or at said injection needle.

38. A method for determining a physical or chemical parameter related to a container or a liquid in the container, in particular the fill level of the liquid in the container, wherein the container has an opening for dispensing the liquid at one end, and wherein a holding device for holding an injection needle for injecting the liquid from the container is disposed at the opening, the method comprising:
ascertaining a measured value in the region of the container, by ascertaining with a detection element located at the opening a detection result dependent on the presence of an injection needle, in particular by ascertaining a capacitance between a metallic detection electrode located at the opening and a further metallic structure arranged in or on the dispensing device;
comparing the detection result with a threshold value and establishing a presence or an absence of an injection needle in the holding device depending on the threshold value comparison;
upon detecting that an injection needle is present, applying a first parameter to the measured value by applying a first computation rule, which supplies, based on the measured value, the parameter value upon the presence of the injection needle in the holding device, and providing the first parameter value thus ascertained as the parameter value; and
upon detecting that the injection needle is absent, applying a second parameter value to the measured value by applying a second computation rule, which supplies, based on the measured value, the parameter value upon the absence of the injection needle in the holding device, and providing the second parameter thus ascertained as the parameter value.

39. The method according to claim 38, wherein:
a first calibration function is predefined, which specifies a relationship between the measured value and the physical or chemical parameter value related to the container or the liquid located therein upon the presence of an injection needle in the holding device;
a second calibration function is predefined, which specifies the relationship between the measured value and the physical or chemical parameter value related to the container or the liquid located therein upon the absence of an injection needle in the holding device; and the method comprises:
upon detecting that the one injection needle is present, applying the first calibration function to the measured value and providing the result thus obtained as the parameter; and
upon detecting that the one injection needle is absent, applying the second calibration function to the measured value and providing the result thus obtained as the parameter value.

40. The method according to claim 38, which comprises using as the measured value a capacitance between two measuring electrodes that are arranged on an exterior of the container.
